(19) United States
(12) Patent Application Publication
(10) Pub. No.: US 2019/0275345 A1
(43) Pub. Date: Sep. 12, 2019

(54) REMOTE CONTROL OF LIGHT-TRIGGERED VIROTHERAPY

(71) Applicants: Academia Sinica, Taipei (TW); National Taiwan University, Taipei (TW)

(72) Inventors: Pan-Chyr Yang, Taipei (TW); S.-Ja Tseng, Taipei (TW); Zi-Xian Liao, Kaohsiung (TW)

(73) Assignees: Academia Sinica, Taipei (TW); National Taiwan University, Taipei (TW)

(21) Appl. No.: 16/347,327

(22) PCT Filed: Nov. 3, 2017
(86) PCT No.: PCT/US2017/059882

§ 371 Date: May 3, 2019

Related U.S. Application Data
(60) Provisional application No. 62/417,946, filed on Nov. 4, 2016.

(51) Int. Cl. A61N 5/06; A61K 47/69; A61K 41/00; A61P 35/00

(52) U.S. Cl. CPC A61N 5/062; A61K 47/6923; A61K 41/00; A61N 2005/0663; A61P 35/00; C12N 2750/14123; C12N 2750/14132; A61K 41/0057

(57) ABSTRACT

Ironized viral particles such as ironized adeno-associated viral particles, which may carry a photosensitizer such as a KillerRed protein, and uses thereof in light-triggered virotherapy against tumor.

### RELATED APPLICATION

This application claims the benefit of the filing date of U.S. Provisional Application 62/417,946, filed on Nov. 4, 2016, the content of which is herein incorporated by reference in its entirety.

### BACKGROUND OF INVENTION

Among innovative treatments for cancer, virotherapy represents a class of promising cancer therapeutics, with viruses from several families currently being evaluated in clinical trials (Bell et al., Cell Host Microbe, 2014; Russell et al., Nat. Biotechnol., 2012; Miest et al., Nat. Rev. Microbiol., 2014). In most clinical trials of virotherapy, patients were treated with virus via intratumoral injection (Miest et al., Nat. Rev. Microbiol., 2014). Enhanced systemic delivery of virus in cancer treatment remains an obstacle in effective virotherapy. (Ledford, Nature, 2015; Bell et al., Cell Host Microbe, 2014; Russell et al., Nat. Biotechnol., 2012; Miest et al., Nat. Rev. Microbiol., 2014; Kotterman et al., Nat. Rev. Genet., 2014). Achieving efficacious and accurate systemic delivery will greatly broaden opportunities in virotherapy.

Clinical trials involving adeno-associated virus (AAV)-mediated gene delivery have enabled successful treatment of a number of monogenic disorders (Kotterman et al., Nat. Rev. Genet., 2014; Naldini, Nature, 2015) and developments in tissue engineering (Yoo et al., Adv. Healthc. Mater., 2016). Directed localization reduces therapeutic dose and consequently lowers risks of AAV-directed immune response, ectopic expression and oncogene activation that leads to mutagenesis. Furthermore, improved approaches to engineering AAV capsids (Lisowski et al., Nature, 2014) and eliminating CpG motifs from the AAV genome (Faust et al., J. Clin. Invest., 2013) have reduced the AAV's immunogenicity by allowing it to avoiding binding to neutralizing antibodies produced from the natural exposure of humans to AAV. Interestingly, AAV capsids engineered to express light-dependent factor motifs bound to a light-switchable protein tagged with a nuclear localization sequence, upon exposure to light, display a significant increase in gene delivery efficiency (Gomez et al., ACS Nano., 2016). However, accurate and specific delivery of genetic material with an appropriate dosage has been a major challenge. For systemically administered viruses, the liver is often the default destination (Kotterman et al., Nat. Rev. Genet., 2014), and represents a barrier when other organs/tissues are the intended targets.

### SUMMARY OF INVENTION

The present disclosure is based, at least in part, the development of magnetic viral particles, for example, ironized viral particles carrying a photosensitizer protein (e.g., a KillerRed protein), which were successfully localized at a site where a magnetic field applies. Such ironized AAV2 particles successfully reduced tumor growth when used in light-triggered virotherapy.

Accordingly, one aspect of the present disclosure features a magnetic viral particle (e.g., an ironized viral particle), comprising a viral particle conjugated to a magnetic oxide nanoparticle (e.g., an iron oxide nanoparticle). The magnetic oxide particle may have a diameter ranging from 1 to 100 nm. In some instances, the magnetic oxide particles may have an average diameter of 5 nm. In some embodiments, the viral particle is an adeno-associated viral (AAV) particle, for example, any of serotypes 1-9 (e.g., AAV2) particle, a lentiviral particle, or an adenoviral particle. In some embodiments, any of the magnetic viral particles described herein may carry a photosensitizer protein such as a KillerRed protein, which may comprise the amino acid sequence of SEQ ID No: 1.

In another aspect, the present disclosure provides a method for treating tumor, comprising: (i) administering to a subject in need thereof an effective amount of a magnetic viral particle as described herein, wherein the magnetic viral particle carries a photosensitizer such as a KillerRed protein; (ii) applying a magnetic field to a tumor site of the subject to induce localization of the magnetic viral particle to the tumor site; and (iii) performing light irradiation on the tumor site of the subject after step (ii). In some embodiments, step (iii) is performed at a wavelength of 540-580 nm (e.g., 561 nm). Alternatively or in addition, the tumor site is at lung, kidney, heart, urinary bladder, skin, breast, or intestine.

In yet another aspect, the present disclosure provides a method for preparing a magnetic viral particle such as an ironized viral particle as described herein, wherein the method comprises chemically conjugating a magnetic oxide nanoparticle (e.g., an iron oxide nanoparticle) to a viral particle in the presence of one or more cross-linking agents. In some embodiments, the chemical conjugation involves ethyl-3-(3-dimethylaminopropyl) carbodiimide hydrochloride (EDC)-mediated conjugation.

In some examples, the preparation method described herein comprises: (i) mixing a magnetic oxide nanoparticle such as an iron oxide nanoparticle with carboxylic acid in the presence of a carboxyl activating agent to form a mixture; (ii) placing a reagent capable of converting a carboxyl group to an amine-reactive NHS ester into the mixture to form the magnetic oxide nanoparticle modified with the amine-reactive NHS ester; and (iii) incubating the modified magnetic oxide nanoparticle with a viral particle to form the magnetic viral particle. The carboxyl activating agent may be a carbodiimide compound, e.g., EDC.

Alternatively or in addition, the reagent in step (ii) is N-hydroxysulfosuccinimide (Sulfo-NHS).

Also within the scope of the present disclosure are (i) pharmaceutical compositions for use in tumor treatment, wherein the pharmaceutical compositions comprise any one of the magnetic viral particles described herein and a pharmaceutically acceptable carrier; and (ii) uses of the magnetic viral particles for manufacturing a medicament for use in tumor treatment.

The details of one or more embodiments of the invention are set forth in the description below. Other features or advantages of the present invention will be apparent from the following drawings and detailed description of several embodiments, and also from the appended claims.

### BRIEF DESCRIPTION OF DRAWINGS

FIG. 1 includes diagrams showing a remote controlled “ironized” virus. Panel A: Concept of a remotely directed Ironized virus by a single tail vein injection for micro-virotherapy. Ironized AAV2 rapidly accumulates in the target tumor site when directed with magnetic field-enforced delivery. Here, KillerRed is expressed by tumor cells infected by AAV2-KillerRed. Light triggers virotherapy. Illumination of KillerRed protein generates ROS and subsequent intracellular damage, promoting cell death. Panel B: Schematic illustration of an exemplary process for conjugating ironized AAV2 with iron oxide nanoparticles using a two-stage conjugation process involving EDC/sulfo-NHS. Photograph shows the transparent yellow solution of Ironized AAV2. Panel C: TEM image of iron oxide nanoparticles with carboxylic acid. Bar=50 nm. Panel D: TEM image of Ironized AAV2 prepared at a molar ratio (1/20) of nanoparticle/EDC showing iron oxide nanoparticles associated with virus. Bar=200 nm. Panel E: Percentages of GFP-expressing cells 6 days post-transduction by Ironized AAV2 for varying molar ratios of nanoparticle/EDC, analyzed by flow cytometry (#, P>0.25; #, P<0.005; based on a two-tailed t test, assuming unequal variances). Data shows mean of measurements conducted in sextuplicate±s.d. Panel F: Viability of HEK293 cells after exposure to Ironized AAV2 at various mole ratios of nanoparticle/EDC. Cell viability is given as the percentage of viable cells remaining after treatment for 24 h, compared against the unexposed cells. Cell numbers were determined by the standard MTS assay (*, P>0.2; **, P>0.5; based on a two-tailed t test, assuming unequal variances). Data shows mean of measurements conducted in sextuplicate±s.d.

FIG. 2 includes photos and diagrams showing remote controlled “Ironized” virus for micro-transduction. Panel A: Representative confocal images of AAV2 distribution over the period of magnetic field exposure (5, 10, or 30 min), immunostained by using Anti-AAV2 antibody and the secondary antibody conjugated to Alexa Fluor® 488. Panel B: Unmodified AAV2 at magnetization for 30 min was used as a control. Bars=1000 μm. Profile curves of fluorescence intensity of GFP-expressing cells infected by Ironized AAV2 (panels C and D) or AAV2 (panel E) incubated with magnetic field exposure (diameter: 1,500 μm) for 30 min and a subsequent 6 days transduction. Images showing the GFP-positive cells infected by Ironized AAV2 or AAV2 were observed by confocal microscopy. All fluorescence intensities from images were assayed by confocal microscopy. The cells were stained by DAPI to label the cell nuclei (adjusted to select red fluorescence). Data shows mean of measurements conducted in sextuplicate. Bars=1,000 μm.

FIG. 3 includes photos and diagrams showing in vitro light-triggered virotherapy. Panel A: The sequence map of pAAV-KillerRed. AAV2-KillerRed was produced by the expression plasmid of pAAV-KillerRed and the packaging plasmids (pHelper and pAAV-RC2). Panels B-D: Profiles of fluorescence intensity of death and nuclei distribution of cells infected by Ironized AAV2 (B and C) or AAV2 (D) after illumination of KillerRed protein. Cells were incubated with a magnetic field (diameter: 1,500 μm) for 30 min subsequent to 6 days when treated with Ironized AAV2 or AAV2 before irradiation. After irradiation for 20 min, the infected cells were observed using Live/Dead® Fixable Far Red Dead Cell Stain Kit. Right panel: representative confocal images show the red fluorescence (cell death). Furthermore, the treated cells were stained by DAPI to reveal the cell nuclei and the confocal images were merged with red fluorescence. All fluorescence intensities from images were assayed by confocal microscopy. Data shows mean of measurements conducted in sextuplicate. Bars=1,000 μm.

FIG. 4 includes diagrams and photos showing in vivo systemic remotely controlled virotherapy. Panel A: Treatment protocols assessing remotely controlled delivery, light-triggered virotherapy using various conditions. Panel B: Tumor growth of various virus-treated EGFR-TKI-resistant H1975 (EGFR^{L858R/T790M}) xenograft tumors via tail vein injection under magnetization (M) and/or light irradiation (L). Tumor sizes were measured by a caliper on the described days (*, P<0.015; **, P<0.001; based on a two-tailed t test, assuming unequal variances). Data shows mean of measurements conducted in sextuplicate±s.e.m. Panels C-F: Representative images of tumor sections from mice per group (n=6) after various treatments on Day 15 were stained with hematoxylin and eosin (H&E) (C), terminal deoxynucleotidyl transferase dUTP nick end labeling (TUNEL) assay (D), DAPI (E), and Prussian Blue (F). Bars=500 m. Panel G: Biochemical analysis of glutamic-oxaloacetic transaminase (GOT), glutamic-pyruvic (GPT), total bilirubin (TBIL), and creatinine (CRE) levels was performed in serum obtained from blood after administration of Ironized AAV2-KillerRed in athymic BALB/c nude mice at Day 0, Day 2, Day 7, and Day 14. Data shows mean of measurements conducted in triplicate±s.d. Panel H: Body weight of mice after various treatments. Body weight of mice were measured at described days in response to the treatments of various formulations by tail-vein injection with and without exposure to M or L. Results show mean of measurements conducted in sextuplicate±s.d. Panel I: Representative IVIS images of mice were taken at Day 14 after tail vein injection of various formulations using AAV2 encoded luciferase as a detect signal. Panel J: Representative IVIS images of organs from mice injected i.v. with various treatments at Day 14.

FIG. 5 includes photos showing localized viral transduction under magnetic field. Panels A-B: Images showing the KillerRed-positive cells infected by Ironized AAV2 (A) or AAV2 (B) as observed by confocal microscopy. The cells were stained by DAPI to label the cell nuclei (blue fluorescence). Bars=1,000 μm.

FIG. 6 includes photos showing tumor growth of mice after various treatments. Representative photograph of H1975 xenograft tumors following various treatments and H1975 tumors excised on Day 15. Bar=1 cm.

FIG. 7 is a photo showing in vivo monitoring of the level of bioluminescence activity. Representative IVIS image of mice were taken at Day 7 after tail vein injection of various formulations using AAV2-luciferase as a detect signal.

FIG. 8 includes photos showing gel electrophoresis and construction of pAAV-KillerRed. Plasmid pAAV-KillerRed was constructed from pKillerRed-dMito and pAAV-MCS. The KillerRed fragment (0.71 kb) was added at the EcoRI and the Sall sites in the KillerRed sequence by using polymerase chain reaction (PCR) with the sequences of primers as described in the Methods section.

FIG. 9 is a schematic illustration showing an exemplary purification and solvent-exchange process of Ironized AAV2 mixture solution. The synthesis of Ironized AAV2 was prepared according to the chemical conjugation in FIG. 1, panel B. After the conjugation reaction, the yellow mixture solution of Ironized AAV2 was purified with a size desalting column (molecular weight cut-offs: 100K) with solvent-exchanged to PBS solution.

FIG. 10 includes diagrams showing determination of relative virus titer. Detection of AAV-KillerRed DNA was determined by reverse transcription PCR (RT-PCR) with the sequences of primers as described in Methods section. The RT-PCR products of AAV2-KillerRed at variant numbers of genome copy (GC) were analyzed by agarose gel electrophoresis (upper panel). The obtained fragments corresponded with the expected size of 539 bp. The standard curve displays the data points for the AAV-KillerRed's GC number and their band intensities obtained by Image J software (lower panel). The RT-PCR samples of Ironized AAV2-KillerRed with unknown content (from FIG. 9) after purification and solvent-exchange were then quantitatively calibrated by the linear regression. Ladder: molecular size marker; NTC: no template control.

### DETAILED DESCRIPTION OF INVENTION

Described herein are magnetic (e.g., ironized) viral particles such as AAV2 viral particles, which were shown to successfully home to a site where a magnetic field applies. Such magnetic viral particles, carrying a photosensitizer protein such as KillerRed, exhibited light-triggered toxicity against tumor when they were homed to a tumor site via the induction of a magnetic field.

### Magnetic Viral Particles and Methods of Making Such

The magnetic viral particles described herein can be any viral particle attached to a magnetic particle such as an iron oxide nanoparticle. In some instances, the viral particle may contain viral proteins encapsulating viral genetic materials (e.g., DNA or RNA depending upon the type of virus), which may facilitate assembly of the viral particles. The viral genetic materials are preferred to be defective as compared with the wild-type counterpart so that the viral particles used in the method described herein cannot self-replicate. Further, the viral particles may be modified such that it cannot infect the native host cells, for example, defective in a viral protein involved in interaction with a cellular receptor. Alternatively, the viral particles described herein are free of viral genetic materials. Such viral particles, also known as viral-like particles (VLPs), can be prepared by methods known in the art. The viral particles can be derived from a suitable virus origin. In some embodiments, the viral particle is derived from a lentivirus, an adenovirus, or an adeno-associated virus.

Magnetic oxide nanoparticles such as iron oxide nanoparticles are magnetic oxide particles (e.g., iron oxide particles). The term “nanoparticles” as used herein refers to, for example, a particle size of 100 nm or less, such as, for example, from about 0.5 nm to about 100 nm, from about 1 nm to about 50 nm, from about 1 nm to about 25 nm, or from about 1 nm to about 10 nm. The particle size refers to the average diameter of the metal particles, which can be determined by a conventional method, such as by TEM (transmission electron microscopy). Generally, a plurality of particle sizes may exist in the metal nanoparticles obtained from the process described herein. In some embodiments, the existence of different sized metal-containing nanoparticles is acceptable.

The magnetic oxide nanoparticles such as iron oxide nanoparticles described herein can have diameters ranging from 1 to 100 nm. In some instances, the iron oxide nanoparticles can be magnetite (Fe₃O₄) or the oxidized form thereof. Ferrite oxide (magnetite) is a naturally occurring mineral, which is widely used in the form of superparamagnetic nanoparticles for diverse biological applications, such as MRI, magnetic separation, and magnetic drug delivery. Mody et al., Applied Nanoscience, 2014, 4(4): pp 385-392. The iron oxide nanoparticles for use in the instant disclosure may have diameters ranging from 1 to 80 nm, e.g., 1 to 60 nm, 1 to 50 nm, 1 to 40 nm, 1 to 30 nm, 1 to 20 nm, 1 to 10 nm, 3 to 10 nm, or 5 to 10 nm. In one particular example, the iron oxide nanoparticles used in the present disclosure have an average diameter of 20 nm, 15 nm, 10 nm, 5 nm, or 2 nm. In some embodiments, the diameters of the iron oxide nanoparticles in the whole population are within 50% (e.g., 40%, 30%, 20%, or 10%) variation of the average diameter.

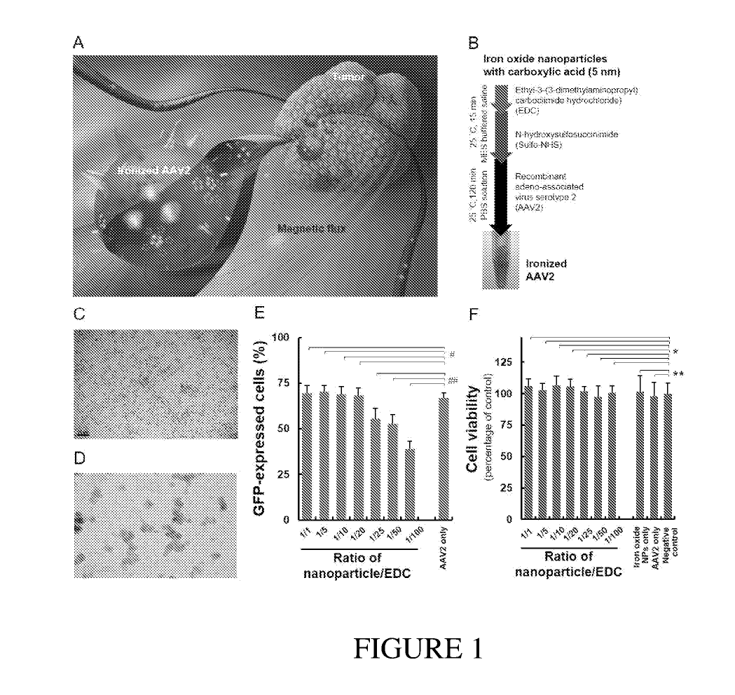

Any conventional method for conjugating magnetic oxide nanoparticles to viral particles (e.g., to viral proteins or viral nucleic acid) can be used to make the magnetic viral particles described herein, using one or more crosslinking agents. Proteins, nucleic acids and drugs can be conjugated to the nanoparticles according to a number of procedures known in the art, such as layer-by-layer with 1-ethyl-3-[3-dimethylaminopropyl]carbodiimide or using 1-ethyl-3-[3-dimethylaminopropyl]carbodiimide with polyethyleneimine. FIG. 1, panel B provides an exemplary illustration of a flowchart for preparing ironized AAV2 viral particles. In this exemplary process, magnetic oxide nanoparticles such as iron oxide nanoparticles can be incubated with carboxylic acid in the presence of a carboxyl activating agent (e.g., EDC) under suitable conditions for a suitable period. A reagent such as N-hydroxysulfosuccinimide (sulfo-NHS) that is capable of converting a carboxyl group to an amine-reactive NHS ester can be added into the reaction mixture, which can be incubated under suitable conditions for a suitable period of time to allow for formation of the magnetic oxide nanoparticles modified with the mini-reactive NHS ester, which would be reactive to certain amino acid side chains of a viral protein to form covalent bonds. The modified magnetic oxide nanoparticles are then be mixed with recombinant viral particles in a suitable solution (e.g., PBS) such as the magnetic oxide nanoparticles are conjugated to the viral particles via chemical conjugation.

The magnetic viral particle described herein may carry a therapeutic agent, which can be encapsulated by the viral particle via conventional methodology. In some examples, the therapeutic agent is a photosensitizer, which is a molecule capable of converting to a cytotoxic agent via a photochemical process, e.g., upon light irradiation. Photosensitizers can be used in photodynamic therapy in treating various diseases, for example, cancer.

In some embodiments, the magnetic viral particle described herein carries a protein-based photosensitizer (photosensitizer protein) such as a phototoxic fluorescent protein. Examples include KillerRed proteins (see, e.g., Fransen et al., Methods Mol. Biol., 1595:165-179; 2017), KillerOrange proteins (see, e.g., Sarkisyan et al., Plos One, 10(12):e0145287; 2015), or Supernova proteins (see, e.g., Takemoto et al., Sci. Rep. 3:2629; 2013). The amino acid sequence of an exemplary KillerRed protein is provided below.

(SEQ ID NO: 1)
MGSEGGPALFQSDMTFKIFIDGEVNGQKFTIVADGSSKFPHGDFNVHAVC
ETGKLPMSWKPICHLIQYGEPFFARYPDGISHFAQECFPEGLSIDRTVRF
ENDGTMTSHHTYELDDTCVVSRITVNCDGFQPDGPIMRDQLVDILPNETH
MFPHGPNAVRQLAFIGFTTADGGLMMGHFDSKMTFNGSRAIEIPGPHFVT
IITKQMRDTSDKRDHVCQREVAYAHSVPRITSAIGSDED

Upon light irradiation, a KillerRed protein can generate reactive oxygen species (ROS), which can be used to kill disease cells, such as cancer cells. The phototoxicity of a KillerRed protein is induced by greenlight irradiation at 540-580 nm and depends on light intensity irradiation time and protein concentration, which can be determined via routine practice.

The KillerRed protein for use in the instant disclosure may share a sequence identity of at least 75% to SEQ ID NO:1 (e.g., 80%, 85%, 90%, 95%, 98%, or 99%) and preserves the phototoxicity activity. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Other exemplary KillerRed proteins and phototoxic fluorescent proteins are known in the art and their amino acid sequences can be obtained from a public gene database, for example, GenBank, using SEQ ID No: 1 as a query. Examples include, but are not limited to, those described under GenBank accession numbers AAY40168, 3A8S_A, 2WIQ_A, BAN81984, 3GB3_A, 4B30_B and 4B30_A. Exemplary KillerOrange proteins can be found in GenBank under accession numbers AQY79141, 4ZFS_A and 4ZBL_A. An exemplary Supernova protein is provided in GenBank under accession number 3WCK_A.

Any of the magnetic viral particles described herein can be mixed with a pharmaceutically acceptable carrier (excipient) to form a pharmaceutical composition for use in the treatment methods also described herein. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), pH adjusting agents (such as inorganic and organic acids and bases), sweetening agents, and flavoring agents.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The pharmaceutical compositions to be used for in vivo administration must be sterile. This is readily accomplished by, for example, filtration through sterile filtration membranes. Therapeutic viral particles compositions are generally placed into a container having a sterile access port, for example, an intravenous solution bag or vial having a stopper pierceable by a hypodermic injection needle.

The pharmaceutical compositions described herein can be in unit dosage forms such as capsules, powders, granules, solutions or suspensions, or suppositories, for, e.g., parenteral administration.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing magnetic viral particles with Intralipid™ or the components thereof (soybean oil, egg phospholipids, glycerol and water).

### Light-Triggered Virotherapy of Tumor Using Magnetic Viral Particles

Magnetic viral particles can be targeted by magnetic fields or used for magnetothermic therapy (Chan (2005); Ito (2005)). Any of the magnetic viral particles, carrying one or more photosensitizers, can be targeted to a desired site (e.g., a tumor site) by magnetic fields. Upon light irradiation, the photosensitizer would produce cytotoxic agents, which would kill diseased cells such as tumor cells at the desired site.

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, or intrathecal routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the pharmaceutical composition containing the magnetic viral particles as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder, such as cancer. A subject having a target disease or disorder can be identified by routine medical examination, e.g., laboratory tests, organ functional tests, CT scans, or ultrasounds. A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder.

Generally, for administration of any of the magnetic viral particles described herein, an initial candidate dosage can be about 2 mg/kg of the photosensitizer contained in the magnetic viral particles. For the purpose of the present disclosure, a typical dosage might range from about any of 0.1 μg/kg to 3 μg/kg to 30 μg/kg to 300 μg/kg to 3 mg/kg, to 30 mg/kg to 100 mg/kg or more, depending on the factors mentioned above. When needed, the magnetic viral particles can be administered repeatedly, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the photosensitizer, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the photosensitizer used) can vary over time.

In some embodiments, for an adult patient of normal weight, doses ranging from about 0.3 to 5.00 mg/kg may be administered. In some examples, the dosage of the photosensitizer in the magnetic viral particles described herein (e.g., KillerRed) can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an photosensitizer as described herein will depend on the specific photosensitizer (or compositions thereof) employed, the type and severity of the disease/disorder, previous therapy, the patient's clinical history and response to the photosensitizer, and the discretion of the attending physician. Typically the clinician will administer a magnetic viral particle comprising the photosensitizer, until a dosage is reached that achieves the desired result. In some embodiments, the desired result is a decrease in thrombosis. Methods of determining whether a dosage resulted in the desired result would be evident to one of skill in the art. Administration of one or more magnetic viral particles carrying the same or different photosensitizers can be continuous or intermittent, depending, for example, upon the recipient's physiological condition, and other factors known to skilled practitioners. The administration of a magnetic viral particle may be essentially continuous over a preselected period of time or may be in a series of spaced dose, e.g., either before, during, or after developing a target disease or disorder.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the magnetic viral particle carrying a photosensitizer and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the photosensitizer, can be dissolved and administered in a pharmaceutical excipient such as Water-for-Injection, 0.9% saline, or 5% glucose solution.

After a magnetic viral particle is administered to a subject in need of the treatment (for example, a cancer patient), a magnetic field can be applied to a desired site of that subject, such as a tumor site, so that the magnetic viral particle would be attracted to the desired site. Exemplary magnets for use to generate a magnetic field include, but are not limited to, electrically charged magnet to deliver an electrical pulse to the desired site and static (not electrically charged) and stationary on the treated area for periods of time to deliver continuous treatment. The magnetic field can be applied to the desired site for a suitable period to allow for homing of the magnetic viral particles to the desired site. Afterwards, a suitable light irradiation can be applied to the same site to trigger the photosensitizer contained in the magnetic viral particles to release cytotoxic molecules to kill diseased cells (e.g., tumor cells). The suitable wavelength, intensity, and time duration of the light irradiation would depend on the type and/or dosage of the photosensitizer (e.g., phototoxic fluorescent protein) used in the treatment. In one example, a KillerRed protein is used and a green light (e.g., at a wavelength of at 540-580 nm) can be used to induce phototoxicity of the KillerRed protein. Other phototoxic fluorescent proteins, including KillerOrange and supernova described above, can also be used.

The light-triggered virotherapy methods described herein may be applied to treat cancer, such as solid tumor. As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

### Kits for Use in Light-Triggered Virotherapy

The present disclosure also provides kits for use in treating diseases/disorders involving diseased cells, such as cancer. Such kits can include one or more containers comprising any of the magnetic viral particles described herein, which carries at least one photosensitizer.

In some embodiments, the kit can comprise instructions for use in accordance with any of the methods described herein. The included instructions can comprise a description of administration of the magnetic viral particle for use in the light-triggered virotherapy against a target disease, such as cancer. The kit may further comprise a description of selecting an individual suitable for treatment based on identifying whether that individual has the target disease. In still other embodiments, the instructions comprise a description of administering a magnetic viral particle to an individual at risk of the target disease.

The instructions relating to the use of a magnetic viral particle and/or the photosensitizer contained therein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a target disease such as cancer. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a magnetic viral particle carrying a photosensitizer as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

Clinical virotherapy has been successfully approved for use in cancer treatment by the US Food and Drug Administration (FDA), however a number of improvements are still sought to more broadly develop virotherapy. A particular challenge is to administer viral therapy systemically and overcome limitations in intra-tumoral injection, especially for complex tumor within sensitive organs. To achieve goal, a recombinant adeno-associated virus serotype 2 (AAV2) chemically conjugated with iron oxide nanoparticles (˜5 nm) was constructed, which showed a remarkable ability to be remotely guided under magnetic field. Transduction is achieved with micro-scale precision. Furthermore, a gene for production of the photosensitive protein KillerRed was introduced into the AAV2 genome to enable photodynamic therapy (PDT); or light-triggered virotherapy. In vivo experiments revealed that magnetic guidance of “ironized” AAV2-KillerRed injected by tail vein in conjunction with PDT significantly decreases the tumor growth via apoptosis. This proof-of-principle demonstrates guided and highly localized microscale, light-triggered virotherapy.

### Materials and Methods

### Materials and Cell Culture.

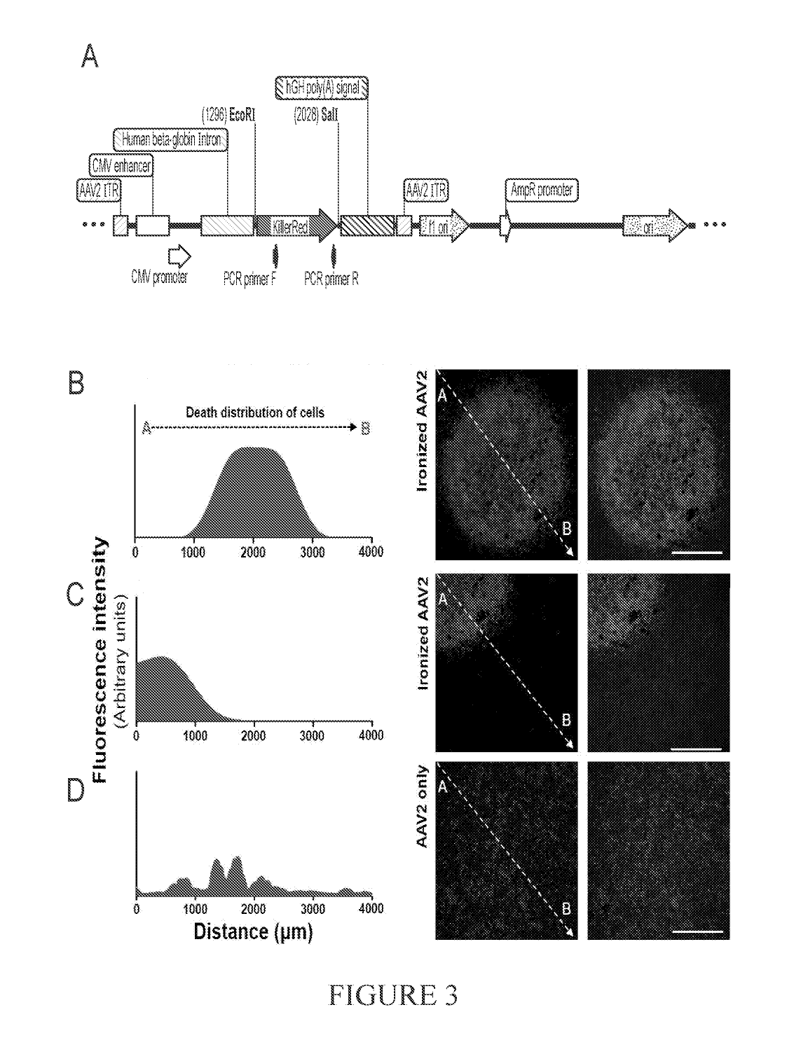

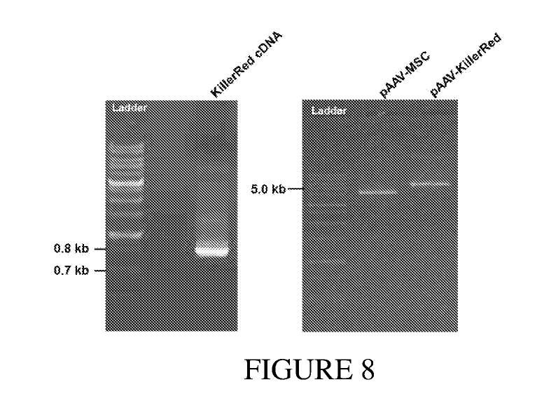

Iron oxide nanoparticles with carboxylic acid (Lot number: 051413A; size: 5 nm; zeta potential: −30 mV to −50 mV) were purchased from Ocean NanoTech (San Diego, Calif.). (1-ethyl-3-(3-dimethylaminopropyl)carbodiimide hydrochloride) (EDC), N-hydroxysulfosuccinimide (Sulfo-NHS), and 2-(N-morpholino)ethanesulfonic acid (MES) buffered saline were purchased from Thermo Scientific Inc. (Rockford, Ill.). Phosphate buffered saline (PBS) was purchased from Sigma Co. (St. Louis, Mo.). Branched polyethyleneimine (PEI, Mw=25,000) was purchased from Aldrich (Milwaukee, Mich.). Plasmid DNA of pKillerRed-dMito was purchased from Evrogen JSC (Moscow, Russia). Virus (AAV2-Luciferase) and plasmid DNAs of pHelper, pAAV-RC2, pAAV-GFP, and pAAV-MCS were purchased from Cell Biolabs (San Diego, Calif.). Plasmid pAAV-KillerRed was constructed as follows. First, the EcoRI and the Sall sites were added into KillerRed fragment from pKillerRed-dMito by using polymerase chain reaction (PCR) with the following sequences of primers: 5′-GGCGAATTCGCCACCATGGGTTCAGAGGGCGGCCCCGCCC-3′ (SEQ ID NO: 5) and 5′-ACGCGTCGACTTAATCCTCGTCGCTACCGATGGCGCTGGT-3′ (SEQ ID NO: 2). The PCR-generated KillerRed cDNA (0.71 kb) was then cloned into the EcoRI-Sall site of pAAV-MCS to yield pAAV-KillerRed (FIG. 3, panel A and FIG. 8).

The human embryonic kidney 293 (HEK293, CRL-1573, ATCC) and 293T (CRL-3216, ATCC) cell lines were cultured in Dulbecco's Modified Eagle's Medium (DMEM) with 10% fetal bovine serum (FBS), 100 U mL⁻¹ penicillin, and 100 μg mL⁻¹ streptomycin. Cells were cultured in a 37° C. incubator with 5% CO₂.

### Production, Purification, and Titration of Virus

AAV2-GFP or AAV2-KillerRed production was performed using the AAV-2 Helper Free Packaging System (Cell Biolabs). Briefly, AAV2-reporter was produced by PEI-mediated co-transfection of plasmid DNAs (pHelper, pAAV-RC2, and pAAV-transgene) in 293T cells. For each 100-mm dish, 293T cells were transfected with 20 μg of pHelper, 10 μg of pAAV-RC2, and pAAV-GFP (or pAAV-KillerRed). The three plasmid DNAs were mixed with 40 μg of PEI in serum-free culture medium and then thoroughly mixed for 30-60 s by vortex mixing and left for at least 20-30 min. Transfection time was only performed for 30 min. Transfected cells were harvested 3 days after transfection. Purification and titration of AAV2-GFP or AAV2-KillerRed were performed according to the protocols of the ViraBind™ AAV Purification Kit (Cell Biolabs) and QuickTiter™ AAV Quantitation Kit (Cell Biolabs) for viral transduction. The number of genome copy (GC) per milliliter of AAV2-GFP or AAV2-KillerRed stock for each batch (8×100-mm dishes) of virus production ranged from 10¹¹ to 10¹². Purified viruses were stored at −80° C. until use.

### Preparation and Characterization of Ironized Virus

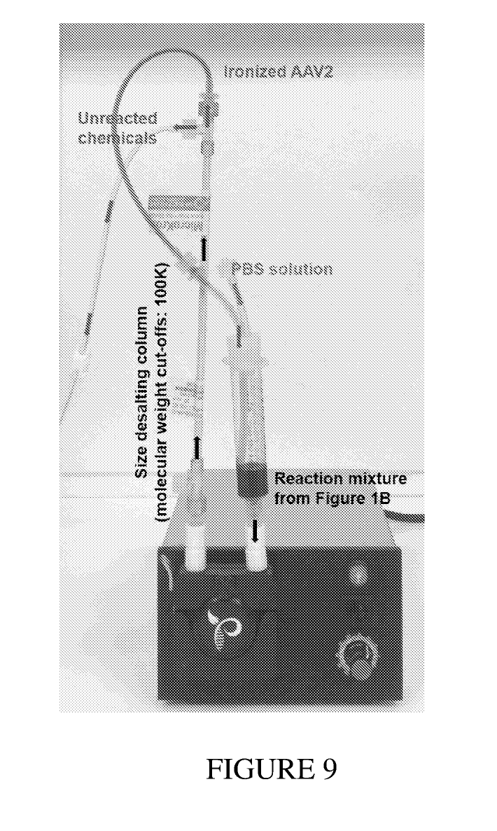

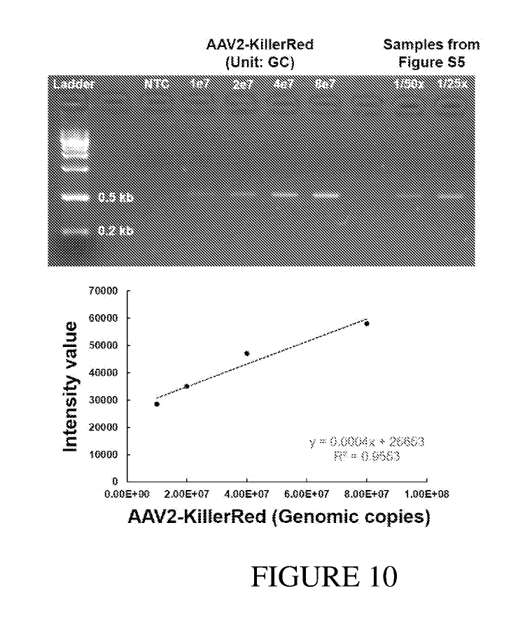

Ironized AAV2 was prepared according to the procedures of chemical conjugation (FIG. 1, panel B). Reaction mixtures were prepared, which contained the iron oxide nanoparticles with carboxylic acid (25 μg, 0.1725 nmol), EDC (0.1725, 0.865, 1.73, 3.46, 4.325, 8.65, or 17.3 nmol) in MES buffered saline solution, and the mixtures had gently added sulfo-NHS with stirring at 25° C. for 15 min to achieve a homogeneous solution of iron oxide nanoparticles with amine-reactive sulfo-NHS ester. The AAV2 stock (0.5 μL, 1×10¹² GC mL⁻¹) in PBS was added dropwise to the mixtures, and then reacted at constant temperature of 25° C. for 120 min. After the chemical conjugation process, the yellow solution was purified by using a size desalting column (molecular weight cut-offs: 100K) that was equilibrated with PBS and solvent-exchanged to PBS (FIG. 9). After the process of purification, a recycle yield of ˜90% was achieved by using PCR with the following sequences of primers for AAV2-KillerRed: 5′-GCCCATGAGCTGGAAGCC-3′ (SEQ ID NO: 3) and 5′-CGATGGCGCTGGTGATGC-3′ (SEQ ID NO: 4); FIG. 10). The obtained PCR fragments of AAV2-KillerRed corresponded with the expected size of 539 bp.

For transmission electron microscopy (TEM, JEOL JEM-1400) analysis, a drop of Ironized AAV2 solution was allowed to air-dry onto a Formvar-carbon-coated 200 mesh copper grid. TEM images were then acquired on a JEOL-1400 microscope operating at an accelerating voltage of 100 kV.

### Transduction

All experiments of viral infection and transduction used culture medium with 10% FBS, 100 U mL⁻¹ penicillin, and 100 μg mL⁻¹ streptomycin. To measure the transduction ability of ironized virus or un-ironized virus without any magnetic fields, AAV2-GFP (green fluorescent protein) was used as the signal indicator. HEK293 cells were seeded in 24-well plates at 1×10⁵ cells well⁻¹ and infected the next day. The test samples of Ironized AAV2 at various molar ratios of nanoparticle/EDC and the AAV2-GFP only were added to cells in DMEM with 10% FBS for 6 days transduction, respectively.

The GFP-expressed cells by viral transduction were quantitatively assessed by flow cytometry (Beckman Coulter, Fullerton, Calif., USA). Cells were detached by 0.025% trypsin and suspensions were transferred to microtubes, fixed by 4% paraformaldehyde. Cells were appropriately gated by forward and side scatter and 10,000 events per sample were collected. The untreated cells were used as the negative control.

### Toxicity of Ironized Virus

7×10⁴ HEK293 cells were seeded in each of the wells of a 24-well plate and fed with culture medium for 12 hours. The cells were then exposed to test Ironized AAV2 at different molar ratios of nanoparticle/EDC and incubated at pH 7.4 for 24 hr. After 24 hr incubation, the transfection media containing test samples were removed. Additionally, the iron oxide nanoparticles or AAV2 was only incubated at pH 7.4 for 24 hr. The CellTiter 96®AQueous one solution cell proliferation assay system (Promega, Madison, Wis., USA) was used to determine the cell proliferation as per previous studies.^{13,16} The optical density (OD) of formazan at 490 nm quantified the cell viability. The reagent contained a tetrazolium compound 3-(4,5-dimethylthiazol-2-yl)-5-(3-carboxymethoxyphenyl)-2-(4-sulfophenyl)-2H-tetrazolium, inner salt (MTS), and the reduction of MTS achieved by untreated cells was set at 100%, and that of test cells was expressed as a percentage of untreated cells.

### Micro-Transduction by an External Magnetic Field

From the results of FIG. 1, panels E and F, the molar ratio of nanoparticle/EDC of 1/20 of Ironized AAV2 for optimized Ironized virus was subsequently selected for the following in vitro and in vivo studies. HEK293 cells were seeded in 35-mm dishes at 2.5×10⁵ cells well⁻¹ and infected the next day. The test samples of Ironized AAV2 or AAV2 only were incubated with cells in DMEM with 10% FBS followed by analysis at different time courses (0, 5, 10, or 30 min) of external magnetic field (2,000-2,200 Gauss) with a diameter of 1,500 μm. Subsequently, samples were fixed by 4% paraformaldehyde (PFA), and the immunostained virus was performed using Anti-AAV antibody (abcam, Cambridge, Mass.) specific to the amino acid 75-86 of major coat protein VP3 of AAV2 for the observation of AAV2 distribution. The signal amplification was developed with Goat Anti-Rabbit IgG H&L conjugated to Alexa Fluor® 488 (abcam) and observed with the confocal microscope. Alternatively, infected cells were observed and analyzed for GFP expression by a confocal microscope after 6 days transduction. The infected cells were stained by DAPI to label the cell nuclei.

As per the previous study of KillerRed activation (Tseng et al., Nat. Commun., 2015), the irradiation time of 20 min was chosen with 561 nm argon laser for optimized ROS generation and KillerRed phototoxicity for the studies. After irradiation of KillerRed, the infected cells were observed using Live/Dead® Fixable Far Red Dead Cell Stain Kit (Thermo Fisher Scientific Inc.) as described by the manufacturer. The treated cells were stained by DAPI to label the cell nuclei.

### Mice Studies

All procedures involving animals were permitted by Academia Sinica Institutional Animal Care and Utilization Committee (AS IACUC). Athymic BALB/c nude mice (6 weeks-old male) were provided by National Laboratory Animal Center (Taiwan). Mice were maintained in a controlled environment with a 12 h/12 h light/dark cycle, housed in groups of 5 maximum and allowed food and water ad libitum.

### In Vivo Light-Triggered Virotherapy Efficacy

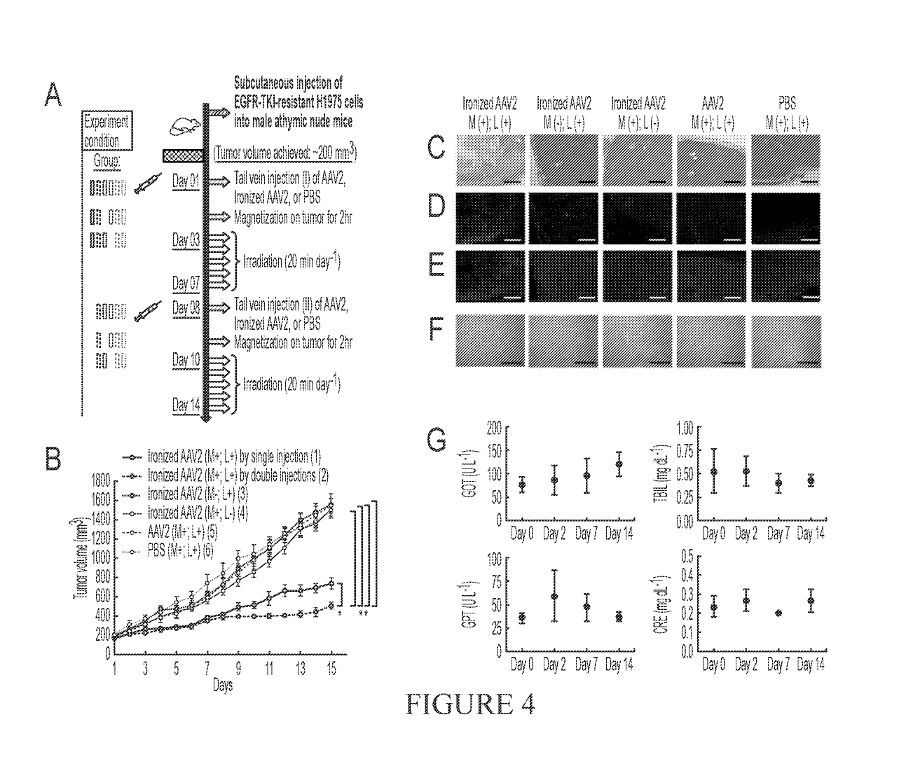

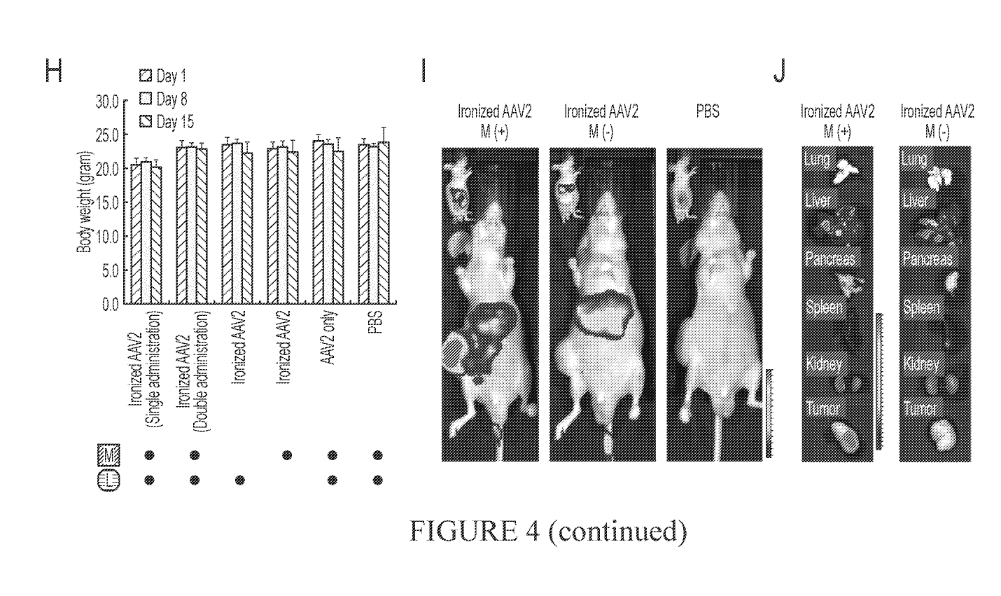

To assess the light-triggered virotherapy effect of Ironized AAV2 or unmodified AAV2, with or without an external magnetic field on the tumor site, EGFR-TKI-resistant H1975 cells, which carries the L858R and T790M, xenograft tumors were established by injecting 2×10⁶ cells subcutaneously into abdomens of 6 week-old male athymic nude mice as prepared according to the trials in FIG. 4, panel A. Once tumors reached ˜200 mm³ volume, mice were randomized into five groups, and tail vein injected with 100 μL of PBS with Ironized AAV2 (5×10⁹ GC mouse⁻¹) or AAV2 (5×10⁹ GC mouse⁻¹). The treatment of PBS injection was used in control mice. In the treatments of applying a magnetic field to targeted tumor, the H1975 (EGFR^{L858R/T790M}) xenograft tumors were exposed to magnetic fields at 1.5 T Gauss for 2 hr.

At Day 3, animals were treated with 1.5 mW mm⁻² total irradiance for KillerRed activation. The tip of the laser fiber was mounted above the tumor, perpendicular to the animal. This regimen was determined following initial optimization experiments (Tseng et al., Nat. Commun., 2015). The laser treatment was administered to the tumor for 20 min every day for 5 days starting from the 3nd day after injection as described in FIG. 4, panel A. Tumor growth was measured every day following treatment using calipers. The length (L) and width (W) of the tumor was measured and the tumor volume was calculated according to the following formula: tumor volume=(0.5 L²)W. Tumor size examination was conducted 24 h after the last treatment.

### Histological and Immunohistochemical Analysis

H1975 (EGFR^{L858R/T790M}) xenograft tumor were harvested at 15 days after innoculation. Harvested xenograft tumor were fixed in 10% formalin, paraffin-embedded, and 5-mm sections were stained with hematoxylin and eosin (H&E) and examined by microscopy. Xenograft tumor sections were also stained with Prussian Blue or Click-iT® Plus TUNEL assay with Alexa Fluor® 594 (Molecular Probes, Eugene, Oreg.) to detect the iron oxide nanoparticle of Ironized AAV2 within tumors or observe the in situ apoptosis detection.

### Blood Analysis

After administration of Ironized AAV2-KillerRed at Day 0, Day 2, Day 7, and Day 14, the blood serum obtained from athymic BALB/c nude mice was collected by using orbital sinus blood sampling. Biochemical analysis of glutamic-oxaloacetic transaminase (GOT), glutamic-pyruvic (GPT), total bilirubin (TBIL), and creatinine (CRE) levels were evaluated. For determination of GOT and GPT levels, the enzymatic method by measuring relative aspartate aminotransferase (AST) and alanine aminotransferase (ALT) activity was used. Also, the level of TBIL, which is an indicator of hepatic cellular damage including hepatoma and hepatitis, was determined by using Randox Diagnostic Test kits according to manufacturer's instructions. As the indicator for kidney function, CRE level was tested by using Randox Diagnostic Test kits.

### In Vivo Bioluminescence Imaging

Ironized AAV2-luciferase or AAV2-luciferase in sterile-filtered PBS solution (100 μL total volume containing 5×10⁹ GC AAV2) was injected via tail vein in mice with or without an external magnetic field on xenograft tumor. Bioluminescence imaging was achieved at Day 7 and Day 14 after treatments. Mice were anesthetized in a chamber filled with 2% isoflurane in oxygen. The luminescent images after intraperitoneal (IP) injection of luciferin (˜240 μL, Caliper Life Sciences Inc., Hopkinton, Mass.) were captured at 5-10 minutes post-incubation by an IVIS imaging system (Xenogen IVIS-50 with Living Image software), with a constant image acquisition time of 5 minutes (Bin: 16/4, FOV: 12). In vivo bioluminescence signals were calculated as the sum of both prone and supine acquisitions for each mouse after background subtraction (photon flux sec-1 cm⁻² sr⁻¹) from a total body region of interest.

### Statistical Analysis

Data are shown as the mean±the standard deviation for experiments performed in sextuplicate. In the measurement of in vivo tumor volume, data are shown as the mean±the standard error of the mean for experiments performed in sextuplicate. In statistical significance testing, P values were calculated using a two-tailed t test, assuming unequal variances.

### Results

(1) Preparation of Iron Suit for Virus.

To validate this new concept rigorously, adeno-associated virus serotype 2 (AAV2) was chemically conjugated with iron oxide nanoparticles with carboxylic acid (size: 5 nm) at various molar ratios of nanoparticle/1-ethyl-3-(3-dimethylaminopropyl)carbodiimide hydrochloride (EDC) through EDC/N-hydroxysulfosuccinimide (Sulfo-NHS) conjugation via the AAV surface proteins' amino groups (FIG. 1, panel B). Transmission electron microscopy (TEM) morphology of iron oxide nanoparticles or Ironized AAV2 had a diameter of ca. 5 nm (FIG. 1, panel C) or 30-40 nm (FIG. 1, panel D).

In vitro characterization and assays for viral infection were undertaken in complete culture medium (10% fetal bovine serum, 100 U mL⁻¹ penicillin, and 100 μg mL⁻¹ streptomycin). To evaluate the effect of chemical conjugation on AAV2 transduction efficiency, an AAV2-GFP (green fluorescent protein) assay was used with flow cytometry (FIG. 1, panel E). At 6 days post-transduction without a magnetic field, cells treated with ironized AAV2 maintained a constant GFP-expression for molar ratios of 1/1 to 1/20 (P>0.25) relative to the control treatment of AAV2. Transduction efficiency dropped to 55.6% (P<0.005) with a molar ratio of 1/25 and to 38.7% (P<0.005) with a molar ratio of 1/100. These data clearly indicate that the nature of the chemical bond used to covalently couple the iron oxide nanoparticles with carboxylic acid to the protein of the AAV2 surface influenced the efficiency of viral transduction due to competition of surface ligands (Lochrie et al., J. Virol., 2006). No cytotoxicity was observed for any of the molar ratios of Ironized AAV2 incubated with Human Embryonic Kidney (HEK293) cells (FIG. 1, panel F). Overall, Ironized AAV2 at the optimized molar ratio of 1/20 was suited to efficiently infect cells by AAV2 with low toxicity for magnetically guided transduction and photosensitization.

(2) Remote Magnetic Control of Ironized AAV2 Distributions

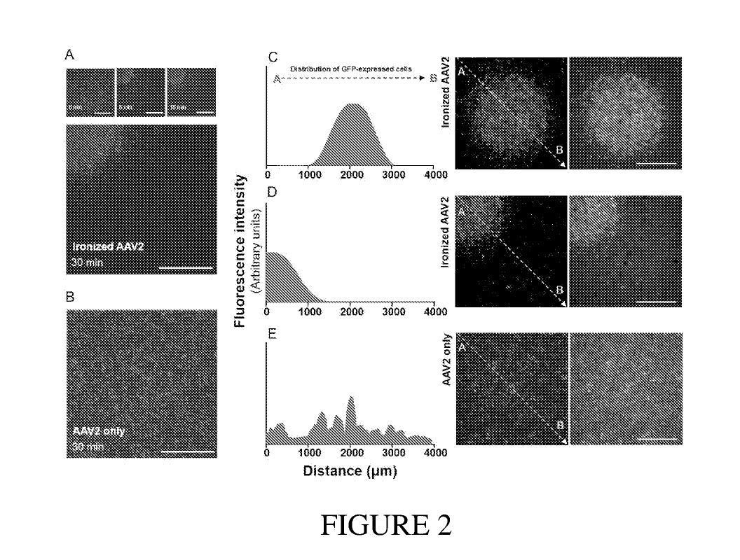

To further evaluate the capability of remote magnetic control of Ironized AAV2, an immunostain assay was utilized with an anti-AAV2 antibody and the secondary antibody conjugated to a fluorescent dye, ALEXA FLUOR® 488, for the observation of AAV2 distribution in cell cultures. Appreciable fluorescence accumulated with 5, 10 or 30 min exposure to a magnetic field (2,000-2,200 Gauss) for producing a localized control of AAV2 distribution (FIG. 2, panel A). In contrast, unmodified AAV2 had a homogenously random distribution with magnetic field exposure for 30 min (FIG. 2, panel B). Likewise, the GFP-expression of cells infected by Ironized AAV2 were examined after treated cells were incubated at 6 days post-transduction after exposure to the same magnetic field with a cylindrical magnet of 1,500 m diameter for a maximum duration of 30 min. The distribution of GFP-expressing cells is represented by the fluorescence intensity in FIG. 2, panels C and D indicating a “micro-transduction” profile 2,000 m in diameter. In contrast, unmodified AAV2 treated-cells expressing GFP were distributed randomly (FIG. 2, panel E).

(3) Light-Triggered Virotherapy Using Ironized AAV2-KillerRed

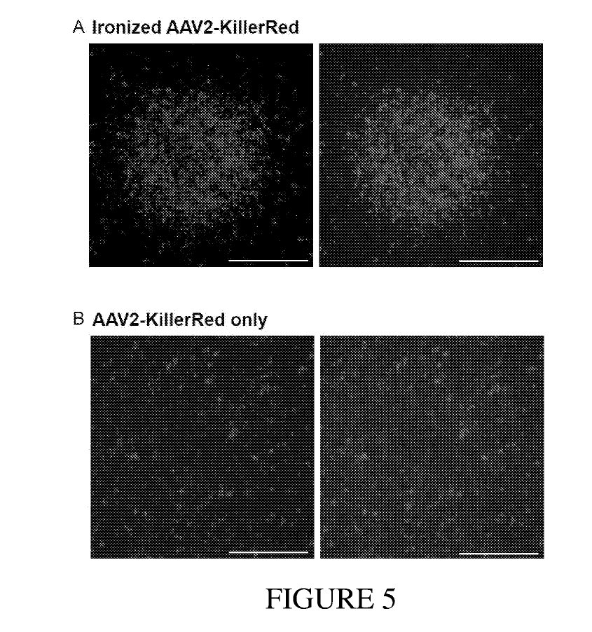

With data confirming successful micro-transduction of cells, light-triggered virotherapy was performed utilizing AAV2-KillerRed (FIG. 3, panel A) with a corresponding wavelength of 561 nm for 20 min irradiation (Tseng et al., Nat. Commun., 2015). Consistent with the observed GFP-expressed cells infected by Ironized AAV2, the KillerRed expression resulted in a circular area only (FIG. 5, panel A). Also consistent with the GFP expression, the AAV2-KillerRed control had no preferential spatial transduction (FIG. 5, panel B). As KillerRed possesses photo-instigated toxicity, cell death was observed after irradiation with yellow light in the cells expressing the KillerRed protein. Distribution of cell death was effectively accumulated in the magnetic field micro-spot and produced no phototoxicity when not infected by AAV2-KillerRed (FIG. 3, panels B and C), demonstrating remotely controlled Ironized AAV2 for light-triggered virotherapy as compared to unmodified AAV2 (FIG. 3, panel D).

(4) Antitumor Activity and Biodistribution in Preclinical Studies Through Bloodstream

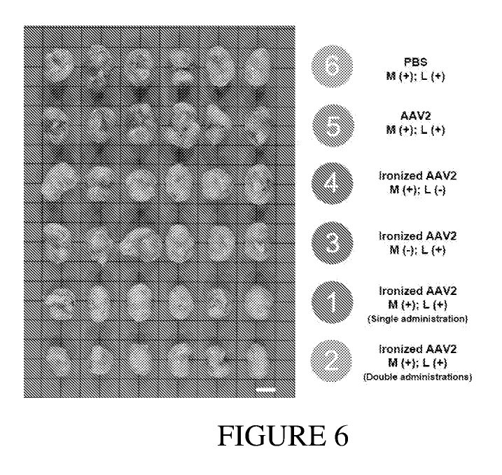

Light-triggered virotherapy treatment were performed using remotely Ironized AAV2-KillerRed in athymic BALB/c nude mice with EGFR-TKI-resistant H1975 (EGFRL858R/T790M) xenograft tumors (FIG. 4, panel A). Notably, treatment with Ironized AAV2-KillerRed was associated with strong suppression of tumor growth (FIG. 4, panel B and FIG. 6), contrasted by a large area of tumor necrosis indicated by H&E (hematoxylin and eosin) staining (FIG. 4, panel C), extensive positive staining by TUNEL (terminal deoxynucleotidyl transferase dUTP nick end labeling) assay (FIG. 4, panel D), and the nucleic acid labeled by DAPI (4′,6-diamidino-2-phenylindole) staining (FIG. 4, panel E) compared with other treatments. Also, the light blue colored area stained with Prussian Blue indicated the distribution and increased presence of iron in the samples exposed to the magnetic field and Ironized AAV2 (FIG. 4, panel F). Single administration of Ironized AAV2-KillerRed injected by tail vein resulted in significantly suppressed tumor outgrowth, however it lacked in long-term suppression. Impressively, when Ironized AAV2-KillerRed was injected again at Day 8, a complete cessation of volume growth was achieved for a further 5 days and was significantly inhibited beyond this (P<0.015). In contrast, delivery of AAV2-KillerRed only or Ironized AAV2-KillerRed without a magnetization field or light irradiation did not result in any statistically relevant anti-tumor effect. Concurrent delivery is consistent with other studies to assist in overcoming the inherently difficult challenge in achieving systemic delivery (Ledford, Nature, 2015; Bell et al., Cell Host Microbe, 2014; Russell et al., Nat. Biotechnol., 2012; Miest et al., Nat. Rev. Microbiol., 2014; Kotterman et al., Nat. Rev. Genet., 2014).

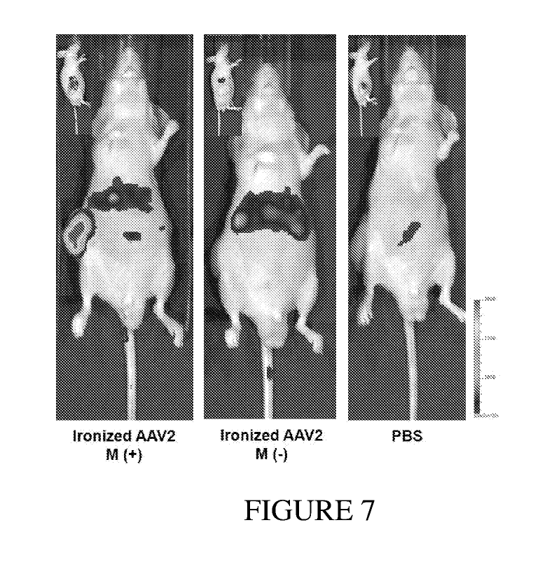

Animals treated with Ironized AAV2-KillerRed were also evaluated for levels of glutamic oxallotransaminase (GOT), pyruvic oxallotransaminase (GPT), total bilirubin (TBIL), and creatinine (CRE) to monitor liver and kidney function. These biochemical analyses did not show any significant liver or kidney toxicity (FIG. 4, panel G). In all experimental groups, no significant loss of body weight was detected, representing a lack of any serious Ironized virus, magnetic field exposure, or light irradiation-related toxicity (FIG. 4, panel H). Biodistribution was considered at Day 7 (FIG. 7) and Day 14 (FIG. 4, panel I) with bioluminescence for animals treated comparably to the in vivo studies but with Ironized AAV2-Luciferase. Consistent with those findings, significant bioluminescence was observed in the tumor at Day 7 and Day 14 when magnetic guidance was utilized and coincides with the tumor suppression shown in FIG. 4, panel B. This reinforces the dynamic dependence upon remotely controlling specificity in delivery. As expected, bioluminescence was also observed in the liver consistent with the clearance pathway of viruses and nanoparticle (FIG. 4, panel J) (Tseng et al., Nat. Commun., 2015).

In summary, specificity in anti-tumor effects with light-triggered virotherapy achieved with remotely guided “Ironized” virus delivery has been demonstrated. Such a technological concept could be harnessed to improve therapeutic efficacy and accuracy with systemic delivery via the bloodstream. There are several distinguishing features of our Ironized AAV2, such as targeted delivery, light-triggered activation of virotherapy, lack of recombination and genomic integration (Kotterman et al., Nat. Rev. Genet., 2014), and strong pre-clinical safety record (Kotterman et al., Nat. Rev. Genet., 2014), that define potential advantages of this concept. Furthermore, magnetic resonance imaging (MRI) instruments can be applied to create pulsed magnetic field gradients in desired direction (Muthana et al., Nat. Commun., 2015), and it may provide the prospect of shaping the accumulation within an internal 3D volume.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A magnetic viral particle, comprising a viral particle conjugated to a magnetic oxide nanoparticle.

2. The magnetic viral particle of claim 1, which is an ironized viral particle comprising the viral particle conjugated to an iron oxide nanoparticle.

3. The magnetic viral particle of claim 1, wherein the magnetic oxide nanoparticle has a diameter ranging from 1 to 100 nm.

4. The magnetic viral particle of claim 3, wherein the magnetic oxide nanoparticle has an average size of 5 nm in diameter.

5. The magnetic viral particle of claim 1, wherein the viral particle is an adeno-associated viral (AAV) particle, a lentiviral particle, or an adenoviral particle.

6. The magnetic viral particle of claim 5, wherein the viral particle is an AAV particle, which is of any one of AAV serotypes 1-9.

7. The magnetic viral particle of claim 1, wherein the viral particle comprises a photosensitizer.

8. The magnetic viral particle of claim 7, wherein the photosensitizer comprises a KillerRed protein.

9. The magnetic viral particle of claim 8, wherein the KillerRed protein comprises the amino acid sequence of SEQ ID NO: 1.

10. A method for treating tumor, comprising:
(i) administering to a subject in need thereof an effective amount of a magnetic viral particle set forth in claim 1, wherein the ironized viral particle carries a photosensitizer;
(ii) applying a magnetic field to a tumor site of the subject to induce localization of the magnetic viral particle to the tumor site; and
(iii) performing light irradiation on the tumor site of the subject after step (ii).

11. The method of claim 10, wherein step (iii) is performed at a wavelength of 540-580 nm.

12. The method of claim 10, wherein the tumor site is at lung, kidney, heart, brain, urinary bladder, skin, breast, or intestine.

13. A method for preparing a magnetic viral particle, comprising chemically conjugating a magnetic oxide nanoparticle to a viral particle in the presence of one or more cross-linking agents.

14. The method of claim 13, wherein the magnetic oxide nanoparticle is an iron oxide particle.

15. The method of claim 13, wherein the chemical conjugation involves ethyl-3-(3-dimethylaminopropyl) carbodiimide hydrochloride (EDC)-mediated conjugation.

16. The method of claim 13, wherein the chemically conjugating step comprises
(i) mixing a magnetic oxide nanoparticle with carboxylic acid in the presence of a carboxyl activating agent to form a mixture;
(ii) placing a reagent capable of converting a carboxyl group to an amine-reactive NHS ester into the mixture to form the magnetic oxide nanoparticle modified with the amine-reactive NHS ester; and
(iii) incubating the modified magnetic oxide nanoparticle with a viral particle to form the magnetic viral particle.

17. The method of claim 16, wherein the carboxyl activating agent is a carbodiimide compound.

18. The method of claim 17, wherein the carbodiimide compound is EDC.

19. The method of claim 15, wherein the reagent in step (ii) is N-hydroxysulfosuccinimide (Sulfo-NHS).

20. The method of claim 13, wherein the viral particle carries a photosensitizer.

21. The method of claim 20, wherein the photosensitizer comprises a KillerRed protein.

22. The method of claim 21, wherein the KillerRed protein comprises the amino acid sequence of SEQ ID No: 1.


SEQUENCE LISTING (5 sequences)

SEQ ID NO: 1 - protein, 239 aa, Artificial Sequence
  Synthetic Polypeptide
MGSEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDED

SEQ ID NO: 2 - DNA, 40 bp, Artificial Sequence
  Synthetic Polynucleotide
acgcgtcgacttaatcctcgtcgctaccgatggcgctggt

SEQ ID NO: 3 - DNA, 18 bp, Artificial Sequence
  Synthetic Polynucleotide
gcccatgagctggaagcc

SEQ ID NO: 4 - DNA, 18 bp, Artificial Sequence
  Synthetic Polynucleotide
cgatggcgctggtgatgc

SEQ ID NO: 5 - DNA, 40 bp, Artificial Sequence
  Synthetic Polynucleotide
ggcgaattcgccaccatgggttcagagggcggccccgccc